(19) United States
(12) Patent Application Publication
(10) Pub. No.: US 2023/0005592 A1
(43) Pub. Date: Jan. 5, 2023

(54) AUTHENTICATION TO MEDICAL DEVICE VIA MOBILE APPLICATION

(71) Applicant: Medtronic, Inc, Minneapolis, MN (US)

(72) Inventors: Daniel Bloomberg, Minneapolis, MN (US); Daniel Isaac George, Minneapolis, MN (US)

(73) Assignee: Medtronic, Inc., Minneapolis, MN (US)

(21) Appl. No.: 17/365,093

(22) Filed: Jul. 1, 2021

(51) Int. Cl. G16H 20/40; G06F 21/31; G16H 20/17; G16H 40/63; A61M 1/16; A61M 1/28

(52) U.S. Cl. CPC G16H 20/40; G06F 21/31; G16H 20/17; G16H 40/63; A61M 1/1601; A61M 1/282; A61M 2205/3561; A61M 2205/3584; A61M 2205/3553; A61M 2205/52; A61M 2205/18

(57) ABSTRACT

A medical system, device, and methods are provided having programming to communicate with a mobile device; the medical device further having programming to authenticate the mobile device; the medical device granting access to one or more functions if the mobile device is authenticated.

### FIELD

Systems and methods are provided for authentication of a device via a mobile application. The systems and methods are related to any apparatus or device requiring security, and more particularly, but not exclusively, to a biomedical device.

### BACKGROUND

Biomedical devices and other therapeutic apparatuses often require user authentication in order to access and use the machines. The devices deliver life-giving or life-supporting therapy to treat diseases or otherwise improve the functioning of the human body. One example of a medical treatment that may be provided by a medical device is dialysis. The devices usually contain sensitive and private medical and health data. Administration of the wrong treatment, or administration of an unauthorized treatment, can have catastrophic life-threatening and legal consequences. To limit access to authorized users, the available systems and methods rely on authentication hardware, tokens, or other custom components such as a radio frequency identification (RFID) card. However, hardware, tokens or other components can be lost and are inconvenient to carry around. Also, the existing systems and methods sometimes require typing in a complicated password.

As such, there is need for quickly and easily providing secured access to patients, doctors, nurses, device administrators, and other authorized persons of a medical device. The need includes authenticating the right people to perform the right operations on the machine at the right time. The need includes configuring the machine, authorizing treatment, setting up treatment parameters, loading data, and administering the treatment using a simple and commonly available device that does not impose additional burdens on a user. The need includes providing a common authentication mechanism that avoids the need for a traditional username/password combination, a hardware token (such as a USB thumb drive with a hardware and/or software key, or an RFID card), biometric authentication, or two-factor authentication. The need includes confirming that a user is authorized to use the machine. The need still further includes loading a correct patient profile with correct information, so that the correct treatment is administered.

### SUMMARY OF THE INVENTION

The first aspect relates to a system. In any embodiment, the system can include a medical apparatus, a transceiver, a processor; and a memory wherein instructions are encoded within the memory to instruct the processor to determine that a mobile device has communicatively coupled to the transceiver, to authenticate the mobile device, and to grant access to one or more functions of the medical device if the authentication is successful.

In any embodiment, the instructions can determine if authentication failed, and can deny access to one or more functions of the medical device.

In any embodiment, the medical device can be a dialysis system.

In any embodiment, the medical device can be selected from a group consisting of: an insulin pump, a sleep apnea machine, a pulse oximeter, an external neural stimulator, intravenous (IV) equipment, a noninvasive blood pressure measuring device, and a smart thermometer.

In any embodiment, the medical device can be programmed to authenticate the mobile device based on a two-factor verification.

In any embodiment, the medical device can be programmed to communicate with the mobile device using either near-field communication (NFC) or Bluetooth low energy (BLE).

In any embodiment, the one or more functions can include initiating a dialysis session.

In any embodiment, the one or more functions can include modifying one or more treatment parameters.

In any embodiment, the one or more functions can access to a function of the medical apparatus.

In any embodiment, the one or more functions can access administrative functions of the medical device, and the instructions can set an expiry for the authentication.

In any embodiment, the one or more functions can include accessing a treatment history.

In any embodiment, the one or more functions can include retrieving one or more patient alerts.

In any embodiment, the one or more functions can include communicating with a patient associated with the medical device.

In any embodiment, the one or more functions can include communicating with a health care professional.

In any embodiment, the one or more functions can be configuring device settings.

In any embodiment, the instructions can determine if the mobile device is associated with a patient of the medical apparatus, and retrieves one or more stored treatment parameters for the patient.

In any embodiment, the treatment parameters can include a parameter selected from the group consisting of treatment time, blood flow rate, dialysis flow rate, sodium prescription, bicarbonate prescription, magnesium prescription, calcium prescription, potassium prescription, glucose prescription, ultrafiltration rate, and patient dry weight.

In any embodiment, the treatment parameters can include a parameter selected from the group consisting of intravenous device type, indicated intravenous drug, drug doses and drug delivery flow rate.

In any embodiment, retrieving stored treatment parameters can include loading the parameters from a local database.

In any embodiment, retrieving stored treatment parameters can include retrieving the parameters from a cloud service.

In any embodiment, retrieving stored treatment parameters can include receiving the parameters from the mobile device via the transceiver.

In any embodiment, a network interface circuit can be communicatively coupled to a cloud service.

In any embodiment, the instructions can also be received from the cloud service with an instruction to authorize a mobile device, and thereafter to authorize the mobile device on the medical device.

In any embodiment, the instructions to authorize the mobile device can include an authorized access level for the mobile device.

In any embodiment, the instructions can include receiving authorization credentials from the cloud service, and storing a local database of authorized users or devices and authorized access levels for the authorized users or devices.

In any embodiment, the instructions can include receiving from the cloud service an instruction to revoke authorization of a mobile device, and thereafter to not authenticate the revoked mobile device.

In any embodiment, the instructions can include storing an expiry for the authorization credentials.

In any embodiment, the instructions can include receiving from a trusted certificate authority cryptographic certificates for one or more mobile devices, and storing the cryptographic certificates in the local database.

In any embodiment, the instructions can include receiving from the certificate authority a certificate revocation for a stored cryptographic certificate.

In any embodiment, authenticating the mobile device can include determining that the mobile device has a certificate issued by the trusted certificate authority and is not expired.

The features disclosed as being part of the first aspect can be in the first aspect, either alone or in combination, or follow any arrangement or permutation of any one or more of the described elements. Similarly, any features disclosed as being part of the first aspect can be in a second aspect described below, either alone or in combination, or follow any arrangement or permutation of any one or more of the described elements.

The second aspect relates to a method performed by a medical device. In any embodiment, the method can include the steps of: receiving authentication information from a mobile device; authenticating the mobile device; and providing access to one or more functions if the mobile device is authenticated.

In any embodiment, the medical device can be a dialysis system.

In any embodiment, the medical device can be selected from a group consisting of: an insulin pump, a sleep apnea machine, a pulse oximeter, an external neural stimulator, and IV equipment.

In any embodiment, the one or more functions can include initiating a dialysis session.

In any embodiment, the one or more functions can include modifying one or more treatment parameters.

In any embodiment, the one or more functions can include accessing a treatment history.

In any embodiment, the one or more functions can include retrieving one or more patient alerts.

In any embodiment, the one or more functions can include communicating with a patient associated with the medical device.

In any embodiment, the one or more functions can include communicating with a health care professional.

The features disclosed as being part of the second aspect can be in the second aspect, either alone or in combination, or follow any arrangement or permutation of any one or more of the described elements. Similarly, any features disclosed as being part of the second aspect can be in the first aspect described below, either alone or in combination, or follow any arrangement or permutation of any one or more of the described elements.

### BRIEF DESCRIPTION OF THE DRAWINGS

FIG. 1 is a system-level view of a medical device ecosystem.

FIG. 2 is a system-level diagram of a medical treatment ecosystem.

FIGS. 3a and 3b are flowchart of a patient mobile application usage workflow.

FIG. 4 is a flowchart of an access privileges workflow.

FIG. 5 is a diagram of cloud service and network connected to a certificate device, a medical device, and a mobile device.

### DETAILED DESCRIPTION

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

The term “authenticate” refers to proving to a device, a system, or a module, to the satisfaction of that device, system, or module, that a person or device is what the person or device purports to be.

The term “administrative functions” refers to functions of an apparatus that can be used to control the apparatus at a low level, or with little or no restrictions.

The term “authentication information” refers to any information or data that can be used to verify the identity of a person or a device. Common examples of authentication information include a personal identification number (PIN), a username and password combination, a biometric, or a two-factor authentication.

The term “Bluetooth low energy (BLE)” refers to a wireless communication protocol that is similar to, but independent of, traditional Bluetooth, that permits devices to communicate over short distances with lower energy. Although BLE is independent of Bluetooth, the two protocols can be supported by a single device, and can use a single antenna.

The term “certificate authority” refers to a cloud or network-based service that issues cryptographic certificates. A trusted certificate authority is a CA that a device trusts to provide valid certificates and certificate data. This could be a well-known public CA, or a special-purpose or local CA setup for a local network.

The term “circuit” refers to a network of electrical or electronic devices, which can be programmable or non-programmable. In the case of a programmable circuit, the circuit also includes any logic or instructions that are used to program the circuit.

The term “cloud service” refers to a service that is provided over a network connection, via a non-local computer.

A “cryptographic certificate” refers a data structure that can be verified via cryptographic methods. For example, the cryptographic certificate can include a cryptographic key of a pre-determined size, which can be verified via a hash algorithm.

The term “database” refers to device stored in either a structured or unstructured data storage format.

The term “dialysis system” refers to any biomedical device that replaces or partially replaces the function of a failed or partially-failed kidney. Dialysis systems can remove urea and other impurities from the blood, and can also remove water and other fluids. Common examples of dialysis include peritoneal dialysis and hemodialysis.

The term “expiry” refers to a relative or absolute date at which a device or thing expires, or is no longer valid.

The term “external neural stimulator” refers to an electrical device to stimulate neurons.

The term “health care professional” refers to any practitioner of the medical arts, including a doctor, a registered nurse, a nurse practitioner, a licensed vocational nurse, or similar.

The term “insulin pump” refers to any medical device that injects insulin into a patient.

The term “local database” refers to data stored locally on a device.

The term “medical device” refers to any electronic device that provides at least one medical function.

The term “mobile device” refers to any handheld computational device. Common examples of mobile devices include cellular phones, smart phones, tablets, and laptops.

The term “near-field communication (NFC)” refers to a set of communication protocols and supporting hardware and software that allow devices to communicate in near proximity to one another. Current NFC standards allow devices to communicate over a distance of approximately 4 cm, or 1½ inches.

The tern “noninvasive blood pressure measuring device” refers to a device to measure a patient's blood pressure that does not require invasive apparatus, such as needles or a large pressure cuff.

The term “patient alert” refers to a notification that can be provided to a patient or to a healthcare professional, for example via a mobile device, to provide any necessary information for the patient or healthcare professional.

The term “pulse oximeter” refers to a device that is generally placed on a finger, and that uses pulses of red light to measure the patient's heart rate and oxygen saturation.

The term “sleep apnea machine” refers to any device that helps to treat or prevent sleep apnea in patients. A common sleep apnea machine is the continuous positive airway pressure (CPAP) device, although other similar devices, such as mechanical devices, can also be used.

The term “smart thermometer” refers to a device for measuring a patient's temperature that also includes communication or network functionality to communicate or coordinate with a home, clinic, or cloud service.

The term “stored patient parameters” refers to information about a patient, which can be stored locally or on a separate device.

The term “transceiver” refers to a circuit that provides transmitter and receiver functionality.

The term “treatment history” refers to a record containing information about a patient's past treatments, including treatment prescriptions, treatment results, and treatment parameters.

The term “treatment parameters” refers to any information that is used to configure or direct a prescribed treatment for a patient. Treatment parameters could include, for example, a prescribed blood flow rate, a prescribed treatment regimen, pharmaceuticals to be administered, pressure settings, minimum or maximum blood pressure, alert conditions, dialysis flow rate, sodium prescription, potassium prescription, glucose prescription, ultrafiltration rate, patient dry weight, intravenous device type, indicated intravenous drug, drug dose, drug delivery flow rate, or other information that can affect the administration of a treatment.

The term “two-factor authentication” refers to any combination of two or more factors that can be used to authenticate a person or a device. A common example of two-factor authentication is a combination of “something you know” (e.g., a password) and “something you have” (e.g., a mobile device, an RFID card, a USB token, a hardware key, a software key, a certificate, or other device or data).

Medical Device Ecosystem

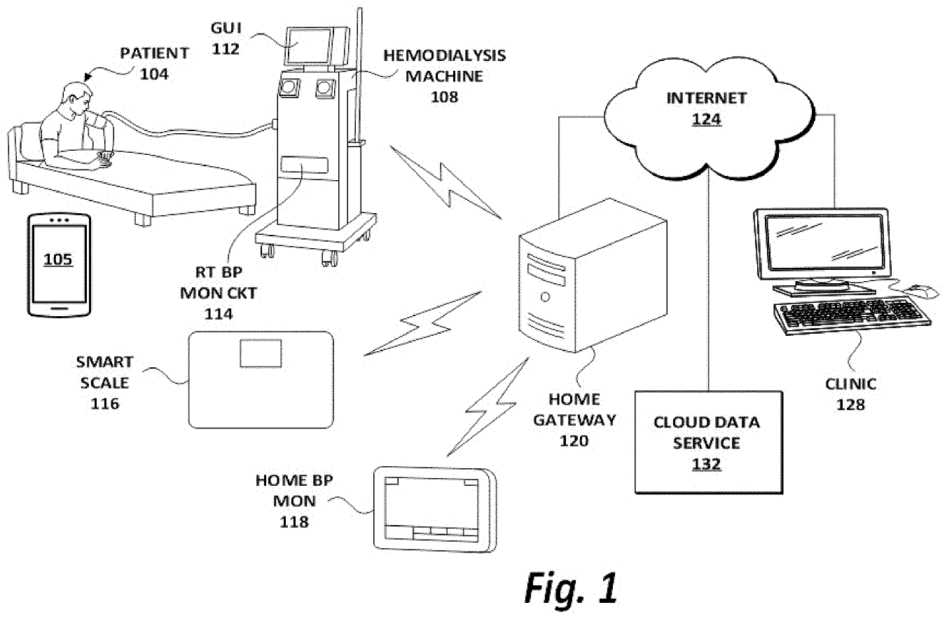

FIG. 1 is a system-level view of a medical device ecosystem 100. Ecosystem 100 can include a home health network, wherein a treatment such as dialysis can be administered by patient 104, or by a caregiver for patient 104. In this context, patient 104 may need to authenticate and/or provide data to a medical device. In any embodiment, the medical device ecosystem 100 can be used to authenticate a patient using a medical device such as a heart-lung machine, CPAP machine, insulin pump, neural stimulator, sleep monitor, intravenous (IV) drug machine, or others medical devices. In one nonlimiting embodiment, the medical device ecosystem 100 can be used to authenticate a user of a hemodialysis or peritoneal dialysis machine that removes toxins such as urea from a patient. In any embodiment, the medical device ecosystem 100 can be used to authenticate a device, including but not limited to a biomedical device, using a mobile device. In any embodiment, the medical device ecosystem 100 can use a mobile device already owned or carried by a user. As such, there is little or no extra burden or load to use the mobile device to authenticate a biomedical device in the medical device ecosystem 100. In some embodiments, a mobile device can include a communication medium, such as Bluetooth (BT), Bluetooth Low Energy (BLE), Near-Field Communication (NFC), WiMax, WiFi, RFID, or similar to communicate with a medical device. In this example, the medical device can have a designated spot, such as an NFC transceiver, that the user can simply touch the phone to, and receive instant authentication via a mobile device installed on the phone.

In any embodiment of the medical device ecosystem 100, a patient 104 operates a medical device such as a hemodialysis or a peritoneal dialysis machine 108, which includes a graphical user interface (GUI) 112. Although FIG. 1 depicts a hemodialysis machine, any machine performing a type of dialysis such as peritoneal dialysis, hemodiafiltration, and ultrafiltration can be used. GUI 112 can permit patient 104 to interact with the machine 108, (as depicted in this particular non-limiting embodiment) and provide inputs and view outputs. In some cases, GUI 112 can include a touch-sensitive display, so that patient 104 can directly interact with GUI 112, via the display. The machine 108 can include various sensors that provide feedback to monitor the success and safety of the dialysis operation. For example, a real-time blood pressure monitoring circuit (RT BP MON) 114 can be provided, so that the patient's blood pressure can be monitored in real-time, or near real-time, and so that the system can detect whether the patient's blood pressure drops dangerously low. Machine 108 can require authentication for certain modes of operation. For example, machine 108 could provide an administrative mode, in which a system administrator can configure the machine. This can require unrestricted access to machine 108. In a therapy configuration mode, a healthcare professional or an authorized agent can configure machine 108 with the prescribed therapy and treatment parameters. Finally, in a therapy administration mode, patient 104 or somebody assisting patient 104 can administer the therapy.

Entering the various modes can be performed by a username and password combination, a biometric authentication, a key card, or a two-factor authentication, by way of illustrative and nonlimiting example. However, operating on the machine can be inconvenient for patient 104 or others who operate on the machine to carry extra devices for such authentication. On the other hand, individuals can carry a mobile device 105 known to one of ordinary skill such as a smart phone or similar. Because patient 104 may already be carrying mobile device 105, use of mobile device 105 can be convenient for authentication to machine 108. For example, mobile device 105 could have a transceiver, such as a BLE or near-field communication (NFC) transceiver. This transceiver can be used to communicate over short distances. Similarly, machine 108 can also have a BLE or NFC circuit, and could, for example, have a designated spot (designated, for example, by a placard or other visible indicator) for performing authentication. Thus, patient 104 can be able to simply touch mobile device 105 to the designated spot on machine 108, and thereby provide instant authentication. This instant authentication can be facilitated by a mobile application running on mobile device 105, which can be provided, for example, by a vendor of machine 108. This simple one-touch authentication uses a device that patient 104 can already have in his or her possession.

In any embodiment, the ecosystem 100 can also include a home gateway 120, which connects to various devices on a network. The various devices can provide additional inputs such as blood pressure and other detectable physiological parameters to machine 108. For example, a smart scale 116 could be used to measure the patient's dry weight, and/or the patient's weight after dialysis. Home blood pressure monitor 118 could be used to periodically check the blood pressure of patient 104, and to keep track of blood pressure trends. In some illustrative embodiments, machine 108, smart scale 116, and/or home blood pressure monitor 118 can communicatively couple to home gateway 120, such as via a wired or wireless network. Home gateway 120 can act as a gateway between the home environment and internet 124, which provides a wider and uncontrolled network. A cloud data service 132 can communicatively couple to home gateway 120 via internet 124, and can provide services to patient 104. The home gateway 120 can provide for communication between the home gateway 120 and a cloud data service 132. The cloud data service 132 can have various software modules to connect to the home gateway 120. Notably, the cloud data service 132 can establish a data communication channel with the home gateway 120 through a TCP/IP protocol or other suitable process, where a terminal is connected to the home gateway 120. The home gateway 120 software can send a first software message to the cloud data service 132 through the data communication channel so that the home gateway 120 establishes connection with cloud data service 132. One of ordinary will understand that other suitable connection methods, steps, and protocols can be implemented to establish such connection with an external cloud server. In some cases, clinic 128 can also connect to cloud data service 132 via internet 124, or via an intranet or other internal network. Clinic 128 can provide data such as electronic health records, a treatment prescription, and other inputs as described herein to cloud data service 132. Cloud data service 132 can then use those data to build the pre-trained artificial intelligence (AI) model for use by machine 108.

Medical Treatment Ecosystem

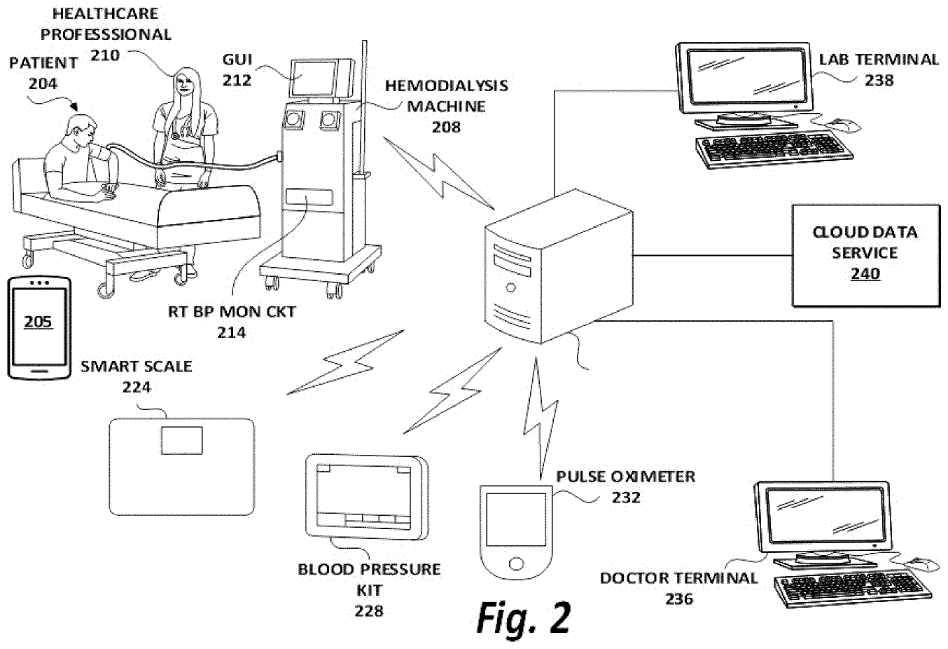

FIG. 2 is a system-level diagram of a medical treatment ecosystem 200. Whereas medical treatment ecosystem 100 of FIG. 1 takes place in a home treatment context, ecosystem 200 can take place in a clinical setting. Thus, patient 204 can be assisted by a healthcare professional 210, who can be, for example, a registered nurse trained in the use of a machine 208, or can be or include a nephrologist, or other healthcare professional. Healthcare professional 210 may, as a general rule, have greater training than patient 204, if patient 204 were required to operate hemodialysis machine 208 individually.

In one nonlimiting embodiment, the machine 208 can be a hemodialysis or peritoneal dialysis machine. The machine 208 can require authentication for certain modes of operation. For example, machine 208 could provide an administrative mode, in which a system administrator can configure the machine. This can require relatively unrestricted access to machine 208. In a therapy configuration mode, a healthcare professional or an authorized agent can configure machine 208 with the prescribed therapy and treatment parameters. Finally, in a therapy administration mode, patient 204 or healthcare professional 210 can administer the therapy.

In any embodiment, entering these various modes can be performed by a username and password combination, a biometric authentication, a key card, or a two-factor authentication, by way of illustrative and nonlimiting example. However, operating on the machine can be inconvenient for patient 204, healthcare professional 210, or others who operate on the machine to carry extra devices for such authentication. On the other hand, individuals sometimes carry a mobile device 205, such as a smart phone or similar. Because patient 204 or healthcare professional 210 can already be carrying mobile device 205, use of mobile device 205 can be convenient for authentication to machine 208. For example, mobile device 205 could have a transceiver, such as a BLE or near-field communication (NFC) transceiver. This transceiver can be used to communicate over short distances. Similarly, machine 208 can also have a BLE or NFC circuit, and could, for example, have a designated spot (designated, for example, by a placard or other visible indicator) for performing authentication. Thus, patient 204 or healthcare professional 210 can be able to simply touch mobile device 205 to the designated spot on machine 208, and thereby provide instant authentication. This instant authentication can be facilitated by a mobile application running on mobile device 205, which can be provided, for example, by a vendor of machine 208. This simple one-touch authentication advantageously uses a device that patient 204 or healthcare professional 210 can already have.

In this example, within the clinic there can be an enterprise gateway 220, which provides connectivity between various devices. Similar to FIG. 1, enterprise gateway 220 connects either via an intranet or via the internet to cloud data service 240. Enterprise gateway 220 can also provide connections to a lab terminal 238 and a doctor terminal 236. A physician can use doctor terminal 236 to input parameters, such as a treatment prescription and other recommendations. Lab terminal 238 can provide lab results, as well as access to electronic health records (EHR) and other data. Cloud data service 240 can be configured to collect and store various inputs. Other inputs can be collected via smart devices around the clinic, such as a smart scale 224, a blood pressure kit 228, and a pulse oximeter 232, by way of illustrative and nonlimiting example. As before, these devices, along with machine 208, can communicatively couple to enterprise gateway 220 via a wired or wireless network. Thus, while healthcare professional 210 is administering treatment to patient 204 via machine 208, healthcare professional 210 can operate GUI 212 to select treatment options. As before, a real-time or near real-time blood pressure monitoring circuit 214 can be provided on machine 208, and can monitor patient 204 to ensure that patient 204's blood pressure does not drop below a danger threshold.

In embodiments, devices can be enrolled by the health care provider, and can be associated with individual users, such as patient 204 or healthcare professional 210. Enterprise gateway 220 can provide (or communicate with) a cloud service, which optionally can maintain communicative contact with machine 208. This allows the healthcare provider to enroll, revoke, authorize, or otherwise manage authorized users and devices. This is different from, for example, a more traditional consumer solution where the end user “owns” his or her own credentials.

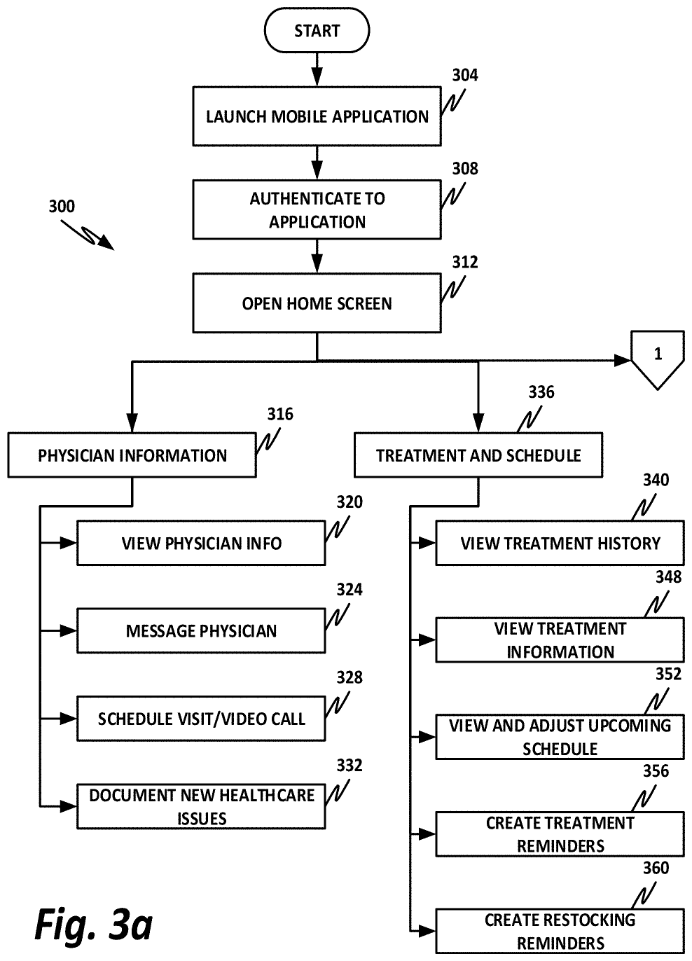

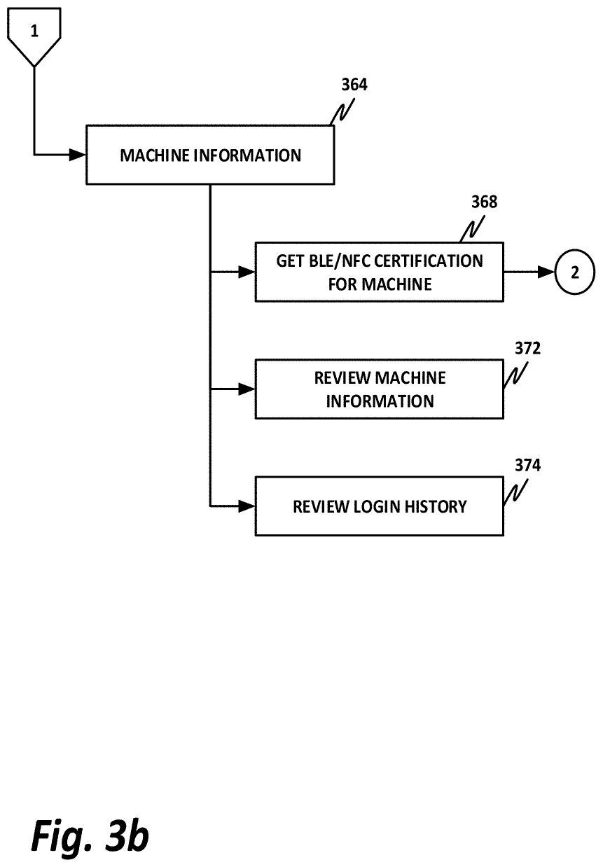

Enrollment can authorize the patient 204 or healthcare professional 210 to use a particular device for therapy, as well as authorizing individual users (via their associated mobile devices) to perform given functions. For example, patient 204 can be authorized to administer (including self-administer) treatment via an authorized device 208, whereas healthcare professionals 210 or administrators can be authorized to perform other functions, as further illustrated in connection with FIGS. 3a and 3b. In any embodiment, authentication can provide mutual assurance. For example, not only can the patient 204 or healthcare professional 210 can be an authorized user, but also the machine 208 can be authorized as the correct device (e.g., programmed with the correct prescription and/or other treatment parameters). Thus, enrollment can include either the machine 208 and the device 208, or both, receiving from enterprise gateway 220 (or some other cloud service) authentication data that associate a user with an authorized mobile device 205 that can be used to identify the user. Machine 208 can also receive (during enrollment, or at some other time) a patient profile, including treatment parameters for patient 204. When mobile device 205 is brought into operational contact with machine 208, machine 208 can also authenticate mobile device 205, determine which user is associated with mobile device 205, ensure that machine 208 is the correct device for that user (e.g., by ensuring that machine 208 includes a profile for patient 204), and then unlock certain functions or features if the authentication is successful.

In any embodiment, device 205 can also help prevent medical errors, by ensuring that the correct patient is being treated. For example, healthcare professional 210 can go through discrete or separate enrollments on a per-patient basis. Authentication can then include healthcare professional 210 accessing a particular patient-specific screen or mode within mobile device 205, tablet of any other suitable hand-held device. This can be done, for example, by using a camera or infrared scanner to scan a bar code on a wrist band worn by patient 204. This can place mobile device 205 in a mode where providing an authentication code that is specific to the combination of patient 204 and medical professional 210. When machine 208 authenticates mobile device 205, device 205 can thus authenticate not only healthcare professional 210, but can also provide a contextual authentication that is unique to the treatment context (e.g., unique to the patient being treated). Device 205 can then ensure that it has a profile loaded for patient 204, and that healthcare professional 210 is authorized to treat patient 204 on machine 208. This can be superior to a more generic authentication that merely determines that healthcare professional 210 is authorized to use machine 208.

Authentication Via Mobile Device

FIGS. 3a and 3b are a flowchart of a method 300 for operating a mobile device according to the system of this specification. In block 304, a user of the mobile device launches the mobile application. The mobile application can be provided, for example, by a vendor of the medical device that is being operated. The application can provide the necessary drivers and communication protocols to carry out the authentication disclosed herein.

In block 308, the user authenticates to the application. For example, the application can require the user to enter a password or a PIN, or to provide another authentication. In common usage, many mobile devices have fingerprint scanners, and users are able to unlock banking, password, and other secure applications by simply placing a finger or thumb on the fingerprint scanner of the mobile device. Thus, the mobile device could use a similar fingerprint scanning technology, thus allowing the user to authenticate to the application using a very natural and common gesture that the user is already familiar with.

In block 312, once the user has authenticated to the application, the user enters a home screen for the application. The home screen for the application could include various fields, such as a physician information field 316, a treatment and scheduling field 336, or (in FIG. 3b) a machine information field.

Physician information field 316 provides access to various physician-related functions. For example, in block 320, the user can be able to view physician information. This can include, for example, the physician's name, phone number, location, links to a map for the clinic, or other information about the physician.

Block 324 provides a facility to message or otherwise contact the physician. Thus, if the patient has questions, or needs to otherwise communicate, the user could operate this control to get in touch with the physician.

Block 328 is a control that allows the patient to schedule a visit or videoconference with the physician. This can be useful, for example, if the patient has questions, or feels that additional clarification is necessary. This could also be used if the patient believes that there can be a need to update a prescription, or otherwise interact directly with the physician.

In block 332, there is a control provided to document newly discovered healthcare issues. For example, if the patient has had an adverse reaction to a treatment or to a drug, or has experienced new symptoms, the patient can notify the attending physician.

Block 336 represents the treatment and scheduling field of the user interface. Within block 336, in block 340, the user can view treatment history. For example, the user could view a graph of past treatments, the results of treatments, and the dates, times, or other information about treatments.

In block 348, the patient can view treatment information. This could include, for example, the prescribed treatment, the length of treatment, therapies administered, pharmaceuticals used, or other information.

In block 352, the patient can view and adjust an upcoming schedule. For example, the patient can view when treatments are to happen, and if there is a conflict, the patient can be able to suggest a replacement time for that treatment.

In block 356, the patient can create or modify treatment reminders. For example, if the patient wants to be reminded 30 minutes before each treatment, the user can set an alert, which can be provided via a known and existing alert application programming interface (API) for the mobile device.

In block 360, the user can also create restocking reminders. For example, if the medical device has consumables, the patient can need to remember to order restocking of these consumable supplies. Thus, the application can use an existing reminder architecture or API within the mobile device to create reminders for the patient.

Turning to FIG. 3b, in block 364, the patient can also view machine information. For example, in block 374, the patient can review login history, such as by viewing times and dates in the past when the patient has logged into the device.

In block 372, the patient can review machine information, such as the device serial number, the device model number, the manufacturer, the state of consumables, and other information about the device.

In block 368, the patient can operate a control to get the BLE/NFC certification for the machine. This control can be the control used for the actual authentication to the device, and can be used to initiate communication. In one example, the mobile application includes a certificate or other security key that can be used to cryptographically verify the identity of the mobile device. Following off-page connector 2, this certificate or security key can be provided via a technology such as BLE or NFC to the medical device for authentication of the mobile device, and consequently, authentication of the operator of the mobile device.

Biomedical Device Authentication

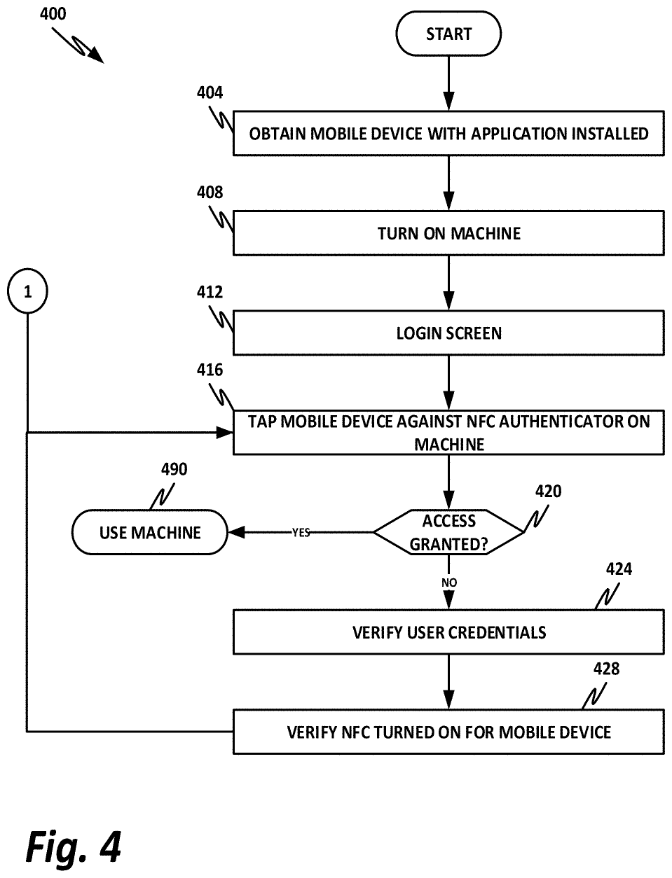

FIG. 4 is a flowchart of a method 400. Method 400 can be performed by the biomedical device that requires authentication to enter certain modes of operation.

In block 404, the patient can obtain a mobile device with the installed application. This mobile device could be configured to perform method 300 of FIG. 3, and examples are illustrated as mobile device 105 of FIG. 1, and mobile device 205 of FIG. 2.

In block 408, the user turns on the biomedical device that is to administer the treatment. In block 412, the user encounters the login screen for the device. As described above, this login screen could provide a traditional login mechanism, such as a username and password combination. Similarly, this login screen could expect an RFID key card, or a USB hardware token. However, as described above, such login mechanisms can be relatively cumbersome and can require inconvenient interactions from the user. Use of a properly configured mobile device for authentication can be preferable, as illustrated by following off-page connector 1 from FIG. 3.

In block 416, the user taps the mobile device against the NFC authenticator on the machine. As described above, this NFC authenticator could be indicated by a placard, an icon, or some other visual indicator representing the spot where the NFC or BLE authentication can take place. The user taps the mobile device against this spot, and the authentication proceeds. The medical device can then open an NFC or BLE session with the mobile device, and establish communication. Once the two devices have established, for example, a secure link, the medical device can retrieve from the mobile device the appropriate key or security token to authenticate. The medical device then cryptographically verifies this key or token, for example, using a previously provisioned certificate on the medical device.

In decision block 420, the medical device determines whether authentication was successful, and if so, in block 490, access is granted.

If authentication was unsuccessful, then this could mean that the device is an unauthorized device, and there will be no authentication. Alternatively, it's possible that the device is authentic, but that some other issue has prevented authentication. Thus, in block 424, the user can verify the user credentials, and in block 428, the user can verify that the NFC transceiver for the mobile device is turned on. After performing these checks, the user can again attempt authentication in block 416.

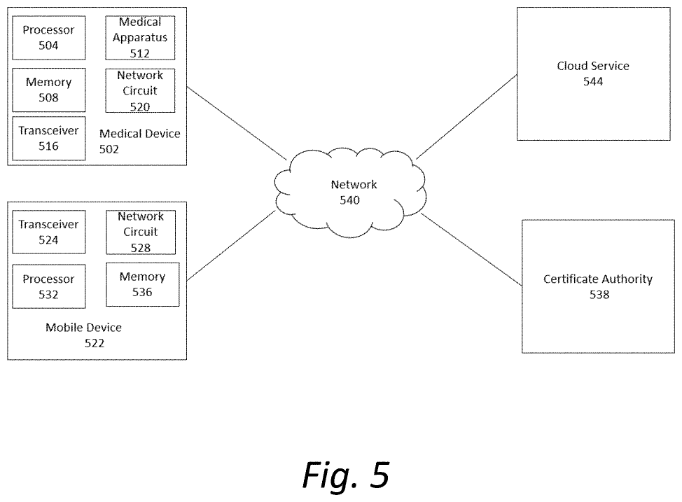

FIG. 5 is a block diagram of selected elements of authentication ecosystem. The authentication ecosystem includes a medical device 502, to which a user can authenticate via a mobile device 522. A network 540 connects medical device 502 and/or mobile device 522 to certain cloud-based services, such as a cloud service 544, and a CA 538.

In this illustrative example, medical device 502 includes a processor 504 and a memory 508. Processor 504 and memory 508 together provide a hardware platform on which software can run. This can include software for authenticating the user, for controlling and configuring the device, for controlling access to the device, for providing a plurality of modes, and for performing other functions.

Medical device 502 includes a medical apparatus 512, which provides a medical function such as, by way of illustrative and nonlimiting example, a dialysis machine, an insulin pump, a sleep apnea machine, a pulse oximeter, an external neural simulator, and intravenous device, a noninvasive blood measurement device, a smart thermometer, or other home healthcare or clinic-based medical apparatus.

Network circuit 520 provides the hardware, software, and/or firmware to communicatively couple medical device 502 to a network 540. Network circuit 520 could provide a wired network such as an ethernet, a wireless network such as WiFi, or some other communication protocol such as USB, FireWire, or other.

Medical device 502 includes a transceiver 516, which can provide local communication via for example WiFi, RF, BTLE, or other. Transceiver 516 enables medical device 502 to make a local connection 518 to mobile device 522.

Mole device 522 includes a processor 532 and memory 536, which can be used to provide a hardware platform for executing software. A transceiver 524 allows mobile device 522 to make a local connection 518 to transceiver 516 of medical device 502. Mobile device 522 includes a network circuit 528, which allows mobile device 522 to communicatively couple to network 540.

In some cases, cloud service 544 provides centralized credentials for users, including their associated mobile devices such as mobile device 522. Cloud service 544 can push authentication data to medical device 502, including providing definitions for a plurality of modes. When a user uses mobile device 522 to authenticate to medical device 502, medical device 502 can authenticate the mobile device, and then grant access to one or more functions of the medical device if authentication is successful. The one or more functions could include, by way of illustrative and nonlimiting example, initiating a dialysis session, modifying treatment parameters, accessing a function of the medical apparatus, accessing administrative functions of the medical device, accessing a treatment history, retrieving one or more patient alerts, communicating with the patient associated with medical device, communicating with the healthcare professional, or configuring device settings.

Alternatively, if authentication fails, then medical device 502 can deny access to the one or more functions of the medical device.

In some embodiments, medical device 502 can determine that mobile device 522 is associated with a patient of the medical apparatus, and can retrieve one or more stored treatment parameters for the patient. The treatment parameters can include, by way of illustrative and nonlimiting example, treatment time, blood flow rate, dialysis flow rate, sodium prescription, bicarbonate prescription, magnesium prescription, calcium prescription, potassium prescription, glucose prescription, ultrafiltration rate, and/or patient dry weight. For an intravenous device, the parameters could include, for example, intravenous device type, indicated intravenous drug, drug dose, and drug delivery flow rate.

Medical device 502 could retrieve these parameters from a local database, or could query cloud service 544 for the treatment parameters. In another embodiment, particularly if medical device 502 lacks always-on network connectivity, medical device 502 could query mobile device 522 via local connection 518 for the parameters. Mobile device 522 could retrieve the treatment parameters from a local database, or from cloud service 544.

Authentication via mobile device 522 can be managed by cloud service 544, and also via CA 538. Certificate authority (CA) 538 can be any trusted certificate authority, which could be a well-known certificate authority that is publicly trusted, or a CA that has established a private trust with cloud service 544, such as a certificate authority operated by cloud service 544 specifically for the benefit of medical device 502 or a class of medical devices similar to medical device 502.

Cloud service 544 can instruct medical device 502 to authorize certain mobile devices, or to not authorize other mobile devices. This authorization could include an expiry, which can in some cases be enforced by a certificate with its own expiry. For example, CA 538 can issue a certificate to mobile device 522, which certificate can have an expiration date. CA 538 can also issue validation data to medical device 502, such as information necessary to authenticate the certificate provided by mobile device 522. Thus, when mobile device 522 authenticates to medical device 502, mobile device 522 can be authenticated only after providing a valid certificate and the certificate has not expired. Furthermore, cloud service 544 can define an authorized access level associated with mobile device 522, or more particularly for a user associated with mobile device 522. For example, if mobile device 522 is owned by an administrative user, then cloud service 544 can instruct mobile device 502 via network 540 to authorize administrative access after medical device 502 has authenticated mobile device 522. Because such administrative access can be both powerful and dangerous, the authorization can include an expiration date.

In some cases, medical device 502 can receive authorization credentials from cloud service 544, and store a local database of authorized users or devices, including authorized access levels for those authorized users and devices. Furthermore, medical device 502 can receive from cloud service 544 instructions to revoke authorization of a mobile device, and thereafter cease authenticated authenticating the revoked mobile device.

One skilled in the art will understand that various combinations and/or modifications and variations can be made in the described systems and methods depending upon the specific needs for operation. Various aspects disclosed herein can be combined in different combinations than the combinations specifically presented in the description and accompanying drawings. Moreover, features illustrated or described as being part of an aspect of the disclosure can be used in the aspect of the disclosure, either alone or in combination, or follow a preferred arrangement of one or more of the described elements. Depending on the example, certain acts or events of any of the processes or methods described herein can be performed in a different sequence, can be added, merged, or left out altogether (e.g., certain described acts or events may not be necessary to carry out the techniques). In addition, while certain aspects of this disclosure are described as performed by a single module or unit for purposes of clarity, the techniques of this disclosure can be performed by a combination of units or modules associated with, for example, a device.

1. A medical device, comprising:
a medical apparatus;
a transceiver;
a processor; and
a memory wherein instructions are encoded within the memory to instruct the processor to determine that a mobile device has communicatively coupled to the transceiver, to authenticate the mobile device, and to grant access to one or more functions of the medical device if the authentication is successful.

2. The medical device of claim 1, wherein the instructions determine if authentication failed, and denying access to one or more functions of the medical device.

3. The medical device of claim 1, wherein the medical device is a dialysis system.

4. The medical device of claim 1, wherein the medical device is selected from a group consisting of: an insulin pump, a sleep apnea machine, a pulse oximeter, an external neural stimulator, intravenous (IV) equipment, a noninvasive blood pressure measuring device, and a smart thermometer.

5. The medical device of claim 1, wherein authenticating the mobile device comprises two-factor verification.

6. The medical device of claim 1, wherein the transceiver is a short-range wireless transceiver.

7. The medical device of claim 6, the transceiver is selected from a near-field communication (NFC) or Bluetooth low energy (BLE) transceiver.

8. The medical device of claim 1, wherein the one or more functions comprise initiating a dialysis session.

9. The medical device of claim 1, wherein the one or more functions comprise modifying one or more treatment parameters.

10. The medical device of claim 1, wherein the one or more functions comprise access to a function of the medical apparatus.

11-31. (canceled)

32. A method performed by a medical device, comprising:
connecting to a mobile device via a transceiver;
receiving authentication information from the mobile device;
authenticating the mobile device; and
providing access to one or more access-controlled functions of the medical device if the mobile device is authenticated.

33. The method of claim 32, wherein connecting to the mobile device comprises connecting wirelessly.

34. The method of claim 32, wherein connecting wirelessly comprises connecting according to a near-field communication (NFC) or Bluetooth low energy (BLE) protocol.

35. The method of claim 32, wherein the medical device is a dialysis system.

36. The method of claim 32, wherein the medical device is selected from a group consisting of: an insulin pump, a sleep apnea machine, a pulse oximeter, an external neural stimulator, and IV equipment.

37. The method of claim 32, wherein the one or more functions comprise initiating a dialysis session.

38. The method of claim 32, wherein the one or more functions comprise modifying one or more treatment parameters.

39. The method of claim 32, wherein the one or more functions comprise accessing a treatment history.

40. The method of claim 32, wherein the one or more functions comprise retrieving one or more patient alerts.

41. The method of claim 32, wherein the one or more functions comprise communicating with a patient associated with the medical device.

42. (canceled)
